(19) United States
(12) Patent Application Publication
(10) Pub. No.: US 2024/0415728 A1
(43) Pub. Date: Dec. 19, 2024

(54) A SYSTEM, METHOD AND GARMENT FOR MONITORING AND CONTROLLING FLUID PRESSURE DURING COMPRESSION THERAPY

(71) Applicant: Arjo IP Holding Aktiebolag, Malmö (SE)

(72) Inventor: Michael David NEWTON, Boerne, TX (US)

(73) Assignee: Arjo IP Holding Aktiebolag, Malmö (SE)

(21) Appl. No.: 18/699,931

(22) PCT Filed: Oct. 11, 2022
(86) PCT No.: PCT/SE2022/050917

§ 371 Date: Apr. 10, 2024

Related U.S. Application Data
(60) Provisional application No. 63/254,325, filed on Oct. 11, 2021.

(51) Int. Cl. A61H 9/00

(52) U.S. Cl. CPC A61H 9/0092; A61H 2201/0188; A61H 2201/1246; A61H 2201/164; A61H 2201/165; A61H 2201/5043; A61H 2201/5048; A61H 2201/5071

(57) ABSTRACT

Compression system for monitoring and controlling fluid pressure during compression therapy has inflatable or deflatable compression garment, an air pump for regulating air pressure to the compression garment when connected to the air pump, an indicator unit, and controller operatively connected to the air pump for monitoring usage of the compression garment. The indicator unit has a first display indicator associated with the time of the monitored usage of the compression garment in a first operation state of the compression system when the compression garment is delivering compression to a patient, and a second display indicator associated with the time of the monitored usage of the compression garment in a second operation state of the compression system when the compression garment is not delivering compression to a patient. The indicator unit automatically swaps between the first and second display indicators associated with the states of operation of the compression system.

### TECHNICAL FIELD

The present invention relates to a compression system, method and garment for monitoring and controlling fluid pressure during compression therapy applied to a patient's limb or other anatomical structure This invention relates to medical compression systems and in particular to pneumatic systems having an inflatable/deflatable article connected to a fluid source, for example a pump. An example use of such a compression system is in the provision of prophylaxis for venous thromboembolism (VTE) or the treatment of other circulatory medical conditions including edema, ischemia and venous insufficiency. The present invention further relates to the display of the time of usage of the compression system and the connected compression garments.

### BACKGROUND

Medical compression systems and in particular to pneumatic systems have an inflatable/deflatable article connected to a fluid source, for example a pump. An example use of such a compression system is in the provision of prophylaxis for venous thromboembolism (VTE) or the treatment of other circulatory medical conditions including edema, ischemia and venous insufficiency.

These systems typically consist of a controller or pump acting as a fluid source and at least one patient-fitted article or garment intended to provide compression to an anatomical area of a patient such as an individual limb or part thereof such as foot, calf, thigh, hand or arm.

It is well known for such systems to have a coupling assembly connecting the article or garment to the fluid source, the coupling assembly comprising a first male insert connector and a cooperating second female receptacle for receiving the first male insert to form a coupled state. The male insert member and the female receptacle both define a pathway for the flow of fluid through to the garment when in the coupled state. The garment may have either the male or the female connector mounted to it. The garment-based connector has an identification component fitted to its connector that is capable of being measured, detected or read by the pump-based connector.

A garment connector known in the prior art (e.g. Newton U.S. Pat. No. 7,398,803) includes an identification component that can be automatically sensed by the compression pump when connected. The use of this component and the associated approach to configure a compression pump automatically through the setup of prophylaxis parameters is also well established in the prior art (e.g. Newton U.S. Pat. No. 6,884,255).

### SUMMARY

The present disclosure relates to a system, method and garment for monitoring and controlling compression therapy to a patient's limb or anatomical structure. With the above description in mind, an aspect of some embodiments of the present invention is to provide increased monitoring and indication to the user on the degree of usage of the system and allows for the active promotion to ensure increased prophylactic use. It seeks to mitigate, alleviate or eliminate one or more of the above-identified deficiencies in the prior art.

An aspect of the present invention relates to a compression system for monitoring and controlling fluid pressure during compression therapy is provided. The system comprises at least one inflatable or deflatable compression garment, an air pump for regulating air pressure to the compression garment when connected to the air pump, an indicator unit and a controller operatively connected to the air pump for monitoring the usage of the at least one compression garment. The indicator unit comprises a first display indicator associated with the time of the monitored usage of the at least one compression garment in a first state of operation of the compression system when the compression garment is delivering compression to a patient, and a second display indicator associated with the time of the monitored usage of the garment in a second state of operation of the compression system when the compression garment is not delivering compression to a patient. The indicator unit is configured to automatically swap between the first and second display indicators associated with the states of operation of the compression system 1.

An aspect of the present invention relates to a compression garment is provided for fitment to a limb or part thereof of a human body, where the duration of connected time is monitored by a system for monitoring and controlling fluid pressure during compression therapy such that the time in a first state of operation is totalized and displayed on a user interface of an air pump of the compression system.

An aspect of the present invention relates to a compression system is provided for monitoring and controlling fluid pressure during compression therapy. The system comprises at least one compression garment, an air source for regulating air pressure to at least one compression garment when connected to the air source, a controller operatively connected to the air source and configured for monitoring the usage of the at least one compression garment, a limb detection unit. The at least one compression garment comprises a connection unit with an identification component, wherein the identification component being made of ferrite, steel or brass. The limb detection unit is configured by the compression system based on the nature of the identification component of the compression garment.

An aspect of the present invention relates to a method is provided for monitoring and controlling fluid pressure during compression therapy in a compression system. The system comprises a pump, at least one inflatable or deflatable compression garment, an indicator unit, a control unit for monitoring the usage of the at least one compression garment. The indicator unit comprises a first display indicator associated with the time of the monitored usage of the at least one compression garment in a first state of operation of the compression system when the compression garment is delivering compression to a patient, and a second display indicator associated with the timed of the monitored usage of the garment in a second state of operation of the compression system when the compression garment is not delivering compression to a patient. The method comprises starting the compression system, identifying connected compression garments, starting prophylaxis operation, detecting and displaying the usage of the at least one compression garment of a compression system and automatically swapping between the first and second display indicators associated with the states of operation of the compression system.

The features of the embodiments and aspects detailed can be combined in any combination.

### BRIEF DESCRIPTION OF THE DRAWINGS

Further objects, features and advantages of the present invention will appear from the following detailed description of the invention, wherein embodiments of the invention will be described in more detail with reference to the accompanying drawings, in which:

FIG. 1 shows a compression system for fluid pressure control according to an embodiment of the present invention,

FIG. 2 shows a control system diagram of the compression system according to an embodiment of the present invention,

FIG. 3a-d shows different embodiments of an indicator unit according to an embodiment of the present invention,

FIG. 4 shows a timing diagram of usage and non-usage of a compression system according to an embodiment of the present invention with the functional aspects of the present invention,

FIG. 5 shows a flowchart of an embodiment of a method for fluid pressure control in a compression system according to the present invention,

### DETAILED DESCRIPTION

Embodiments of the present invention will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference signs refer to like elements throughout.

This invention relates to medical compression systems and in particular to pneumatic systems having an inflatable/deflatable garment connected to a fluid source, for example a pump. An example use of such a compression system is in the provision of prophylaxis for venous thromboembolism (VTE) or the treatment of other circulatory medical conditions including edema, ischemia and venous insufficiency.

The present invention further extends the use of the identification component to allow for the timing of the garment connection to the pump and the configuration and selection of parameters associated with the means of accurate detection of the patient limb within the garment.

The use of different identification component types, characteristics, sizes and positions for different garment types allows for the determination and selection of multiple parameters by the compression system. These parameters can provide useful information to a compression system including; specific garment type, model or size of connected garment, number of garments fitted to the pump, as well as a means of confirming which particular techniques and parameters from a plurality of sensing techniques and possible parameters should be used to confirm patient limb presence with the specific garment connected. This approach allows the optimal means to be selected from a plurality of means and employed to detect when the patient's limb is within or encased by the compression garment.

A further aspect of the invention also involves the monitoring of connector engagement using the continued and timed presence of a specific identification component hence measuring for how long the connection has been successfully made. The pump can therefore detect, measure and record the delivery of the compression therapy to the garments through the connector and to the limb within.

The confirmation of garment fluidic connection and the systems use with a patient limb are combined to form an indicated totalized time value forming a first display state on the user interface. This provides an accurate measurement of the duration of delivered therapy/prophylaxis to the patient and creates information that can be used clinically.

A further aspect involves the timing of how long the garment connector has been disconnected during the intended use of the compression system, the time value forming a second display state shown on the user interface. This is particularly beneficial as it provides the clinician with an indication of the lack of prophylaxis due to garment disconnection, which can have significant hazardous clinical implications.

A further aspect of the invention involves the detection of when the garment is physically connected to a controller of the pump, is receiving stimulus via a valid fluidic connection, but the garment has actually been removed from the limb or anatomical structure of the patient and hence not providing the intended clinical effect.

This monitoring can employ a variety of sensing techniques, algorithms and calculations that can differ for each individual garment type and size and uses the identification component present in the garment connector to select the appropriate monitoring configuration.

This aspect further involves combining both the detection of the identification component to confirm fluidic connection and also the identification component is used to configure a sensing means used to identify the presence, removal, reapplication or lack of presence of the garment on the patient anatomical structure. This detection is further timed and forms part of an alternative second state of display of the user interface, This situation can occur, for example, when the patient, carer or clinician removes the compression garment and forgets to replace them, resulting in an equally clinically hazardous situation for the patient. It is particularly beneficial for a clinician to be able to readily see exactly how long the prophylaxis has not been applied so an appropriate clinical response can be followed. The second state of display of the user interface can be formed from minutes, hours or combinations of both.

The integration of the aforementioned monitoring and identification processes with the underlying operation of the compression system therefore provides for a more informative and hence effective compression solution.

The aforementioned integration within the present invention also avoids the difficulties experienced when utilizing alternative garment identification approaches that use only pressure-based sensing methods. Variation can be present in this pressure measurement technique due to many factors including garment fitting, tightness to the limb, sizes and differing limb sizes. These variations can affect the accuracy of any garment identification detection method or the confirmation of a limb within the garment.

The invention includes monitoring of the usage of the product in a clinical setting. This is based on a combination of the following individual aspects:
time-based monitoring and recording of the totalized duration of pump operation with the selected garment(s) including the recording of the duration of pump operation when the requisite number of limbs is identified as being present in the connected garments during their inflation. The recording being in the form of a number of records in the form of start/stop time-based data records as well as incrementing elapsed time counters. This provides a set of records with a time-base to allow the system to determine when and for how long and how effectively the prophylaxis is applied or not applied for,
   combined with at least one of the following key elements
an indication to the user (via a user interface such as the pump LCD and external LED indicators) during pump operation of the overall elapsed time that the prophylaxis is being successfully delivered to the patient anatomical structure located in the connected garment(s). This elapsed time is accumulated over individual prophylaxis episodes and can be reset/cleared for new patients or at the start of each shift or day or as otherwise required. The reset process being able to be undertaken automatically after a prescribed period of non-operation or alternatively by manual intervention by the user via the interface.
an indication to the user (via a user interface such as the pump LCD and external LED indicators) during pump operation of the overall elapsed time that the prophylaxis is not being successfully delivered to the patient due to either the lack of a sensed anatomical structure located in the connected garment(s) or a disconnected garment. This time is shown as a current duration of lack of delivered prophylaxis.
Either of the aforemention durations are visibly indicated during normal controller/pump operation on the user interface and does not require an operator action to access the information via a subsequent screen or menu. This immediately visible display allows the usage information to be seen at a glance by an clinician/carer when passing by the pump without any intervention required and this reduces the clinician workload in the monitoring of the use of the compression system.

It is known that simple timers associated with elapsed use can be included in many products including compression pumps-such as a simple elapsed time since compression was previously started. A novel aspect of the present invention is the automatic changing of the displayed information between a first display state with accumulated usage data comprising previously delivered prophylaxis timing information to a second display state with the current no-usage timing information. The second display state specifically shows the elapsed time associated with the lack of delivery to the patient when the compression system is running and this is presented in the form of a count-up time value, initially shown in minutes. The display of this time is also associated with other user feedback means such as a visual indicator using LCD icons or LED indicators or with an optional audible annunciation.

A specific aspect of the invention involves flashing a LCD leg graphic icon to clearly highlight that a garment on a particular pump output is not providing prophylaxis to a limb—the issue being related to the lack of presence of a limb in the garment rather than any problem with the garment or pump operation. It is an aspect of the invention that the prophylaxis is continually applied to the garment even if it is sensed that no limb or anatomical structure is sensed, this ensures that continued beneficial operation occurs even if a sensing fault occurs.

A further aspect of the invention involves the user display automatically switching from the second display mode back to the first display mode when an anatomical structure is sensed as returning to the garment.

A further aspect of the invention is that the elapsed usage timer is not specifically not incremented when no anatomical structure sensed as being present in association with at least one garment/

One or several of the below listed elements can be incorporated:
Inclusion of a Real Time Clock (RTC) device in the compression system can provide beneficial details such as the actual date and time of day of the system usage. The daily care of the patient can affect or preclude the use of a compression system (e.g. bathing/bathroom visits/medical examinations/diagnostic procedures/medication etc.) and this time is often scheduled or otherwise known and hence recorded in the patient care records. Through the use of the system, including the exact timing of the start and stop of delivered prophylaxis it allows for improvements in nursing management, audit processes and clinical reporting of delivered VTE prophylaxis effectiveness for a patient to be undertaken.
The RTC also allows for a time-stamped record to be stored within the controller memory in the form of a log of the individual events of use versus events and duration of not being used using a start/stop timestamp based measurement technique. This includes time associated with the pump being taken out of use by clinician activity such as garment removal from the pump and the pump being physically switched off. Hence individual patient usage episodes are identified and recorded for later analysis and reporting to the clinician as well as inclusion in the patient's electronic medical record (EMR).

An aspect of the invention is to use both start and stop timestamps-rather than simply recording the elapsed timed duration of the episodes. From this basic data it is possible to build up a detailed and relevant time log of the use of the system and the delivered prophylaxis.

Instead of a simple compliance timer, as is well known in the prior art, a usage monitor and timer that identifies episodes of use-based on detecting limb presence and hence delivered prophylaxis is provided. This timer does not rely on a simple totalization of time over a period but instead it seeks to identify individual periods of compression and durations of use and non-use This approach offers a more useful clinical data function than compliance monitoring based on a simple overall totalized time.

In an aspect which involves the pump displaying the elapsed time since the patient removed the garments from their limbs or unplugged the garment to pump connector(s)—both of which are possible without requiring access to the pump controls. It is this type of incorrect patient-initiated activity that the invention monitors and it is believed to be a major cause of insufficient prophylaxis time. A real life example of this situation is where a patient removes the garment(s) from their limbs in order to visit a bathroom but fails to reapply the garment(s) to the limb when they return.

In this situation, the compression system changes from the first display state to show the second display state which will show the time since the removal occurred. This aspect allows the clinician to readily ascertain the lack of use/compliance to the prescribed care and make a clinical determination on the safe and appropriate intervention for the patient.

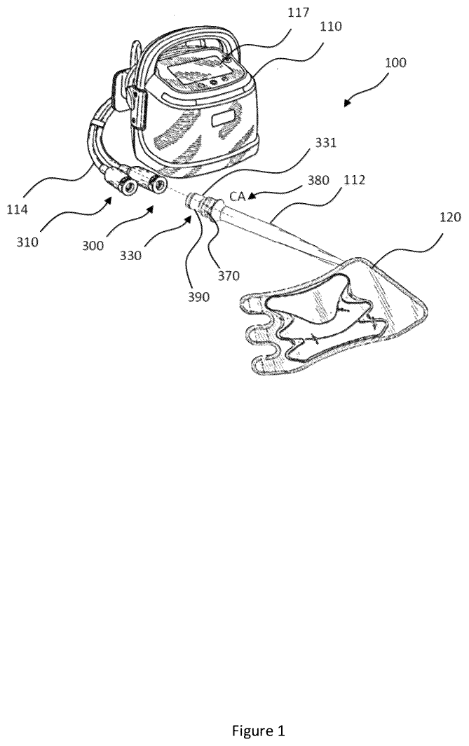

FIG. 1 shows the general arrangement of a compression system 100 for monitoring and controlling fluid pressure during compression therapy. In one embodiment, the system 100 is a gas pressure compression system such as a pneumatic compression system or is based on any type of suitable fluid for the application with inflatable/deflatable articles or garment 120.

The system 100 comprises at least one inflatable/deflatable garment 120 in the form of a compression garment, an air source 110, i.e. an air pump, and a controller 210 (not shown in FIG. 1) operatively connected to the pump 110. The controller 210 is operatively connected to the pump 110 for controlling said pump 110. The pump 110 may be a pneumatic pump.

In an embodiment, the pump 110 is arranged to control fluid flow to and from the inflatable/deflatable garment 120. Accordingly, the pump 110 is arranged to inflate or deflate the inflatable/deflatable garment 120.

The system 100 further comprises a coupling assembly 300 for fluidically connecting the inflatable/deflatable garment 120 and the pump 110. The coupling assembly 300 comprises a connector 330 and a connecting member 310.

The connector 330 is connectable to the connecting member 310 to upon engagement form a fluid pathway through the connector 330 and the connecting member 310. Accordingly, the connector 330 may be connectable to the connecting member 310 to allow for fluid communication through the coupling assembly 300.

The coupling assembly 300 comprises an identification component 390. The position, size and type of the identification component 390 is provided on the connector 330 or the connecting member 310 and a sensing arrangement (not shown in FIG. 1) for detecting the position of the identification component 390 relative to said sensing arrangement during engagement between said connector 330 and connecting member 310.

Thus, a missing connection or partial engagement between the connecting member 310 and the connector 330 may be detectable by means of the detection of the presence and the monitoring of the position of the identification component 390. Accordingly, the risk and impact for misconnection is reduced as through this monitoring and indication.

Engagement between the connector 330 and the connecting member 310 herein refers to a state wherein the connector 330 and the connecting member 310 are in contact. In said state, the connector and the connecting member may be connected, non-connected or partially connected.

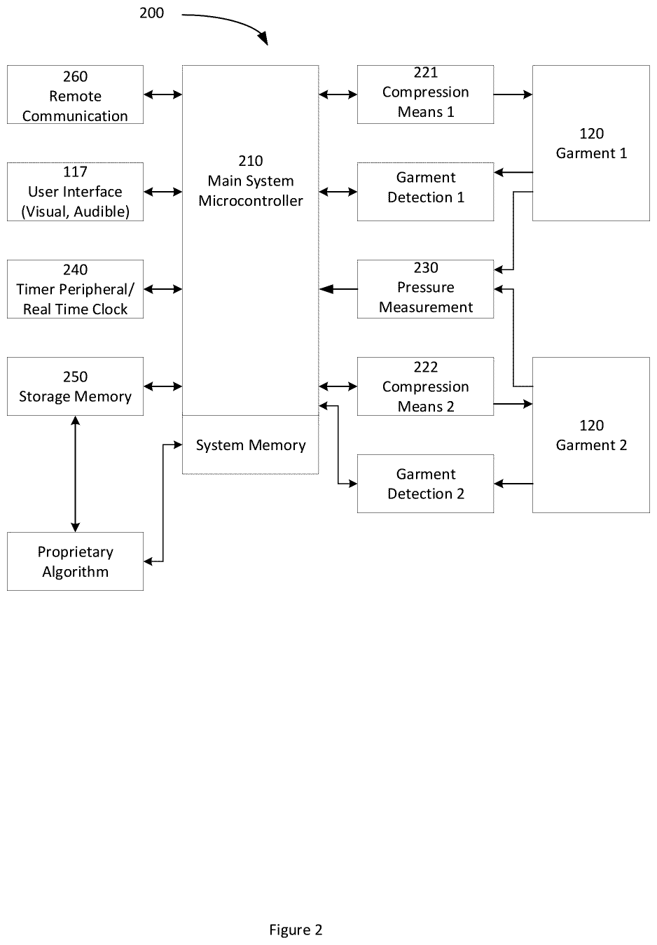

The compression system 100, shown in FIG. 1 in the form of an example format comprising a pump 110, a control system 200, as shown in FIG. 2, a user interface 117, i.e. an indicator unit, comprising visual and audible elements, at a least one compression garment 120 for mounting on a patient's limb, the compression garment being attachable to at least one connecting means 114 via connector arrangements 300, comprising separable physical connector parts 310, 330. The connector part on the garment 330 having a body 331 including an identification component 390 such that the controller 221 of the pump 110 can detect the position, size and type of the at least one connected garment from amongst a plurality of different types. The controller 221 of the pump 110 is configured to be able to monitor the use of the compression garment 120 when in use and when not in use by an individual patient and provide time-based metrics and analysis of its status.

FIG. 2 shows a logical arrangement of elements of the control system 200 of the compression system 100 comprising the controller 210, i.e main system microcontroller, located internally and providing software-based functions. With respect to FIG. 2, the arrangement of the control aspect is shown together with individual functional and logical elements. These elements are shown as individual elements for ease of explanation but it is within the scope of the invention that these can be physically combined in some embodiments.

In an embodiment, at least one garment type provided for attaching to an anatomical structure of a patients anatomy, for example on a limb such as foot, calf or thigh. The garment 120 provides the compression into the patient anatomy directly through its inflation. Each connected garment 120 is sensed via its identification component 390 and this allows the main system microcontroller 210 to identify and configure the required compression for each connected garment based on its detected type.

Feedback is provided by the compression means 221, 222 to the main system microcontroller 210 on the degree of engagement with the anatomy and the effect of the compression. Compression is monitored by the main system microcontroller using a pressure measurement element 230 and this allows the real time control and delivery of the compression from the system 100 to the garment 120. Alternatively, feedback and reporting from the fluidic source 110 (e.g. an air compressor) can be used to detect the operating conditions and ensure consistent delivery of the compression to the anatomical area.

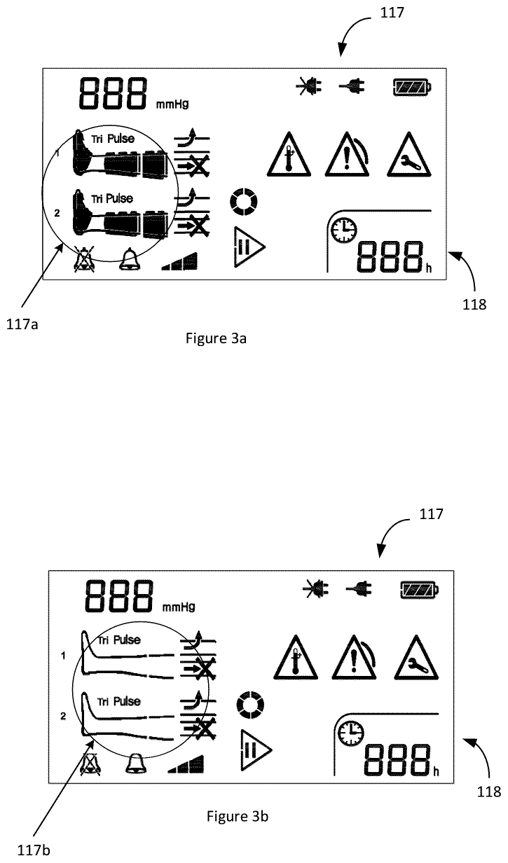

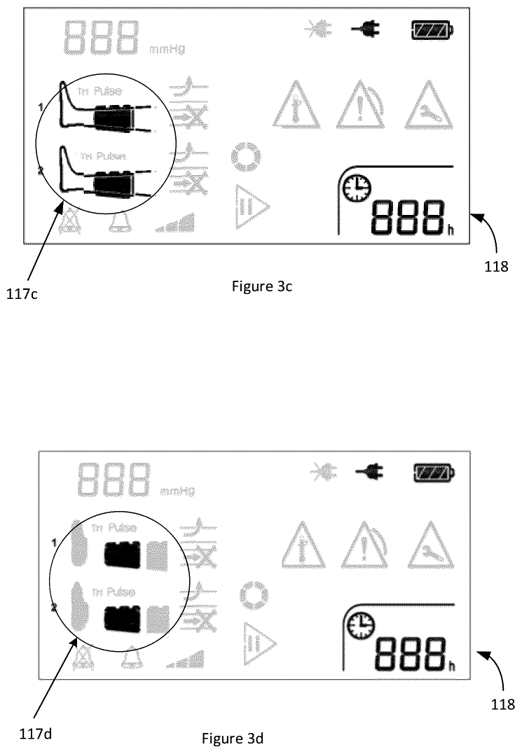

The main system microcontroller 210 can provide visual feedback to the user on the status of the compression system 100 via a user interface 117, 117a-d consisting of visual indications, such as a LCD display, as shown in FIG. 1 and in detail in FIG. 3a-d and/or with other LED based indicators located as part of the compression system. The user interface 117 can also provide audible feedback to the user using speakers, piezo sounders or other forms of audible devices. The user interface 117 can also provide for the control of the compression system 100 through the selection of system operation, operating modes, settings and other user-selectable features via the use of buttons, keypads and other forms of user inputs. FIG. 3a-d shows an example graphical user interface of a compression system known in the prior art in the form of a LCD panel with a full set of available graphic icons displayed. This provides example indications of various functions and state of the operation of the compression system during different modes of operation and at different times.

A timing element 240 is provided that can be used to provide time-based information such as timing duration, clock and calendar information that can be used by the operating software and be stored in the system memory.

A storage memory element 250 is provided that allows the main system microcontroller 210 to store data and records associated with both periods of system usage (i.e. that intended and prescribed) as well as periods of non-usage (i.e. contrary to that intended or prescribed) and includes timing information provided by the timing element. This allows a non-volatile storage of data associated with a typical period of patient care (e.g. lasting over hours and days) as well as data associated with the usage of the pump with multiple patients (e.g. over weeks and months). This data can be accessed that can be analyzed to provide information on the effectiveness of the system use by staff within a healthcare facility.

The main system microcontroller 210 is able to communicate this usage information from the storage memory 250 to external devices using remote communication element 260 (e.g. wirelessly using Bluetooth or Wi-Fi or using a wired connection such as USB).

Further referencing to FIG. 1, the system 100 further comprises an indicator unit 117, a user interface. The indicator unit 117 is operatively connected to the main system microcontroller 210, i.e. controller. The indicator unit 117 is configured to provide an indication to a user based on the position signal.

In an embodiment, the indicator unit 117 is provided on the pump 110. Accordingly, the indictor unit 117 is mounted to the casing of the pump 110. In one embodiment, the indictor unit can also be mounted on the connecting member 310. Thus, the user is provided an indication while operating the pump 110.

In one embodiment, the indictor unit 117 may be a display unit, such as an LCD-display.

In an aspect, the indictor unit includes LED indicators where the LED state of illumination is dependent on the LCD status of first display state and the second display state.

In a further aspect, the LED-based indictor unit changes a constant illumination state to a flashing illumination state based on the LCD status.

In an embodiment, the indictor unit 117 is configured to provide an indication to a user in response to the distal part 331 being in an intermediate position along the connection axis, CA, between the coupled position and the non-coupled position.

In one embodiment, the controller 210 is provided on or within the pump 110. As shown in FIG. 3a-d, the indictor unit 117 is shown in the form of a graphical LCD display with a complete range of icons associated with the various different functions associated with the compression system 100.

FIG. 3b highlights the specific icons from the panel shown in FIG. 3a used for the display of the usage state of the system when no garments are connected, i.e. prior to the use by a patient. This represents the display when the product is not in prophylactic use. The icons showing usage monitoring invention. There are other icons as well, but they are removed for clarity of explanation. The indictor unit 117 shows no connected garments are currently sensed as being connected to the pump. The totalized previous usage of the system is shown in the right hand lower corner 118 of the indictor unit 117 in the form of a three digit elapsed time meter that can be used to indicate durations of hours and minutes. The display image also includes multiple graphic icons of sections of an example limb for each compressive output of the compression system.

FIG. 3c shows an example display of the compression system 100 when the system has detected the connection of two garments intended for connection to a patients legs, specifically on the calf. A set of connected garment icons is indicated and the usage information shown on the right hand lower corner 118 of the display in the form of a three digit elapsed time meter and is associated with the continued use of these garments. FIG. 3c shows only those icons from FIG. 3a that are associated with the indication of a first display state associated with usage monitoring with a patient. This comprises icons associated with an anatomical structure such as a limb with foot, leg and calf, various garment icons mounted on areas of the limb and a timing display including a three digit and seven segment numerical display. The nature of the garment icons displayed depends on those detected by the garment detection means. It is within the scope of the invention that different icons are used for different garment types. The timing information shown on the timing display 118 is associated with the monitored use of the connected garments.

FIG. 3d shows an example second state display of the compression system operating with the same connected calf garments as detailed in FIG. 3c but where the patient's limbs are not present. The usage information shown on the right hand lower corner 118 of the display in the form of a three digit elapsed time meter and the timing information is changed to be associated with the lack of use of these garments.

An aspect of the invention is that the indictor unit 117 involves a flashing limb effect alternating between the limb icons shown circled in FIGS. 3c and 3d when in the second display state. The alternation of the icons being in the form of flashing or change in display contrast level.

Hence, this condition is where the patient limbs have been sensed as not being present and the compression system is not delivering the prescribed therapy or prophylaxis. The timing information shown on the timing display is associated with the monitored lack use of the connected garments.

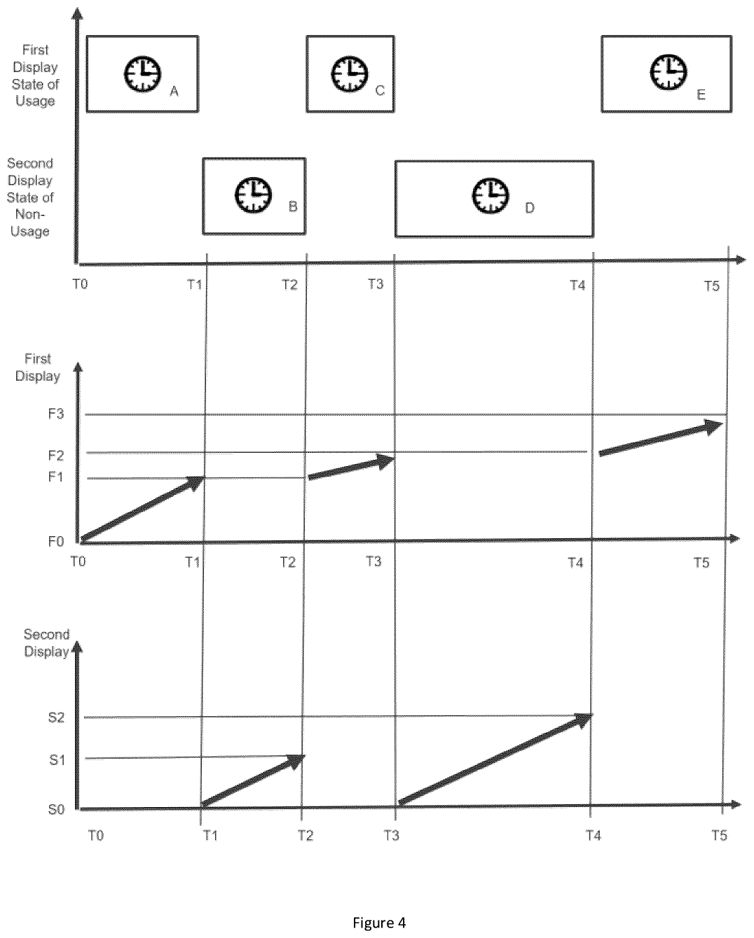

With respect to FIG. 4, this shows pictorially an example typical usage scenario of the compression system of FIG. 1 with a patient. The figure graphically shows the periods of use and non-use and the effect on the first display state and the second display state provided by the seven segment display on the LCD.

This usage scenario involves individual periods of usage (A, C, E) which are interleaved with periods of non-usage (B, D). These periods can last for minutes or hours within the care of a typical patient and are typical of that experienced in a hospital environment. Examples of periods of usage A, C, E include operation of the compression system when the patient is located in a hospital bed, during surgery or sleeping and the compression system is specifically required and intended to be in use. Examples of typical non-usage periods B, D include when the patient is attending a diagnostic procedure (e.g. X-ray) or having personal hygiene (bathing/toileting) and the compression system is specifically not intended to be in use.

A further and important consideration is that the periods of non-usage can occur during the time that the system is actually intended to be used. Examples of this scenario are due to the physical removal of the garments from the patient's limb for an examination then the caregiver failing to replace them. In this scenario the patient is at risk as they are not receiving the prescribed therapy. This aspect is specifically monitored by the compression system 100.

The timings are described accordingly. The first state of usage A lasts for a time from T0 to T1, the duration of which being T1−T0, the LCD display provides a first display state with a timing value increasing during T0 to T1.

This is followed by period of non-usage B, where the second display state is shown. This is followed by a period of usage C where the first display state is shown, a further period of non-usage D with the second display and a final period of usage E with the first display being active.

The occurrence and duration of these individual periods of usage and non-usage is specific to each patient and so the resulting metrics provided by the usage monitoring and performance monitoring function of the compression systems can provide a means of quantifying this.

With reference to FIG. 4, the upper display shows an illustrative example of the present invention of a compression system with two defined states of usage or operation. The first state of usage being associated with the intended normal operation with a patient, the second state being associated with the operation of the system in an unintended mode.

A number of discrete usage episodes are shown in a chronological sequence A, B, C, D, E where episodes A, C and E involve the correct operation of the system delivering compression therapy to a patient as intended and prescribed. Episodes B, D denote examples of usage where the compression is not delivered as intended or prescribed. Many factors can result in the situation shown in episodes B, D such as removal of compression garments from the limb of a patient or disconnection of the garments from the pump or detected fault conditions such as leaks.

Each episode is monitored and timed as shown in FIG. 4.

During the first state of usage, the compression system 100 provides a first type of display to the user. The first type of display includes an incrementing elapsed time that is the accumulated duration of operation in the first state across multiple usage episodes (for example A, C, E). This time is monitored and shown in the form of a delivered prophylaxis time, for example initially in minutes only and then after 60 minutes has elapsed this changes to hours and minutes.

During the second state of usage, the compression system provides a different and second type of display to the user. The second display is in the form of an incrementing time associated with the time spent in the second state of the specific use episode. The time is shown initially in minutes only but then is shown in hours & minutes.

In a further aspect, the second time can be accumulated from each episode of non-use.

It is an advantage of the use of the two displays that the user is only presented with information that is relevant to the state if the compression system. This promotes usability and ease of understanding of the timing information provided.

When the compression system usage changes from the first state to the second state of operation it automatically removes the first state display type and instead only shows the second state display type.

When the usage of compression system changes from the second state of operation to the first state of operation then the compression system automatically reverts to providing only the first state display type.

The timings of the episodes are shown as in FIG. 4 and Table 1, together with a mathematical expression of the displayed values associated with the times for each display type based on the elapsed time ‘t’ in FIG. 4.

TABLE 1
Calculation basis for the displayed timings

| | Episode | Episode | Episode | Episode | Episode |
|---|---|---|---|---|---|
| | A | B | C | D | E |
| State of | First | Second | First | Second | First |
| Usage | | | | | |
| Duration | (T1-T0) | (T2-T1) | (T3-T2) | (T4-T3) | (T5-T4) |
| First | T | N/A | (t-T2) + | N/A | (t-T4) + |
| Display | | | (T1-T0) | | (T3-T2) + |
| State | | | | | (T1-T0) |
| Second | N/A | ‘t’ in | N/A | ‘t’ in | N/A |
| Display | | range 0 to | | range 0 to | |
| State | | (T2-T1) | | (T4-T3) | |

The above table 1 shows that the first display state is only available when the pump is being used and the compressions system is validly providing prophylaxis to the patient, whereas the second display state is only provided when the compression system is not delivering prophylaxis.

In the first display state, the elapsed usage timing information increases during Episode A, then increases through Episode C and then again increases through Episode E to provide a totalized value over the duration of patient care. The first display state is not available during the non-usage episodes where the elapsed timing values does not increment.

This situation is also shown pictorially in FIG. 4.

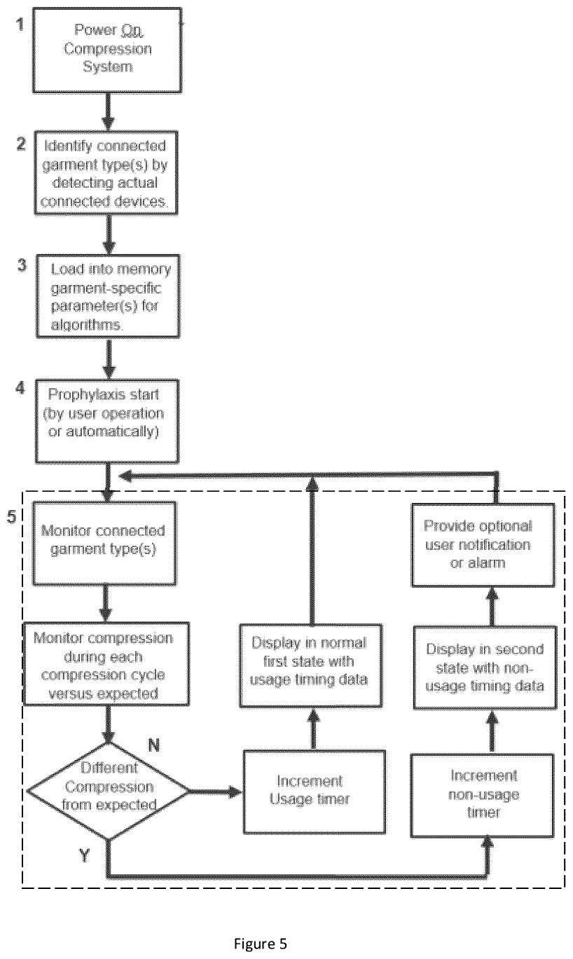

FIG. 5 shows a flowchart detailing an example embodiment of the invention that allows the determination of which display state to provide on the compression system. With regard to FIG. 5, the compression system monitors each connected garment and controls the inflation of the garment in real time and is able to sense the presence of a limb within each compression garment during each inflation. This is achieved though analysis of multiple parameters and readings taken at different times during the inflation process. These readings can include at least one of; the pressure in the connected tubing 114, 112, the pressure in the connected inflated garment 120, connected garment type and the compressor parameters involved with actually providing that specific inflation condition (such as amplitude of compressor drive waveform, frequency of applied drive waveform, current, voltage).

A measurement of prophylaxis delivery is created by combining the above parameters and is compared with expected readings for that specific connected garment type as well as that achieved on the previous inflation of that connected garment. The system looks for significant difference between these two analysis points and uses this difference to determine limb presence.

The compression system is able to change interchangeably between first and second display states based on the measurement of the presence of the limb in the garment. This change of display states occur with operation of only a single connected garment or with two connected garments. The change of display states occur if either of both out of two connected garments is detected as not being used with a patient's limb.

It is within the scope of the invention that additional parameters can be added to the algorithm, and additional outputs can be generated by the algorithm.

When the compression system senses that it is not delivering prophylaxis to a patient it can adapt its indication to the user. The user indication can involve LCD-based indications, LED based indications and audio indications based from the pump. Hence, the priority and level of user indication can be adapted as needed in a clinical setting. This can include a phased and progressive indication that is proportional or associated with the duration of the non-usage episode. Hence, a low level of indication can be provided to the user if the lack of usage has been of a short duration but this can be enhanced as the duration increases and the situation becomes more serious.

In a preferred embodiment, in both of the first and second display states, a flashing green LED indication is provided at multiple locations on the compression system when a duration of 10 compression cycles have been sensed without correct prophylaxis being delivered. The use of multiple LED indicators on a compression system allows for multiple viewing directions ensuring that the state of operation of the compression system and its usage can be readily determined by clinical staff from a variety of directions without having to approach or interact with the compression system.

This LED indication can be further increased in terms of its priority as the non-used time increases, for example though the use of a yellow LED alarm indication instead of a green LED. This change occurs if the number of cycles without the correct delivery of prophylaxis reaches for example 10 cycles.

It is also within the scope of the invention that the indications and timing data provided by the usage monitoring display on the compression system can be replicated at other locations physically away from the compression system.

The compression system can optionally include a means of communication of its status to a remote facility such as central monitoring equipment, a computer, tablet device, telephone or another device capable of displaying the status of at least one the compression system. This communication means could utilize wireless technology (such as Bluetooth, ZigBee or Wi-Fi) and/or a dedicated wired connection to another device capable of displaying the information itself or providing onward communication such as a hospital bed or other medical device such as a support surface/mattress pump.

In an aspect, a compression system 100 for monitoring and controlling fluid pressure during compression therapy is provided. The system comprises at least one inflatable or deflatable compression garment 120, an air pump 110 for regulating air pressure to the compression garment when connected to the air pump, an indicator unit 117 and a controller 221 operatively connected to the air pump 110 for monitoring the usage of the at least one compression garment 120. The indicator unit 117 comprises a first display indicator 117a, 117c associated with the time of the monitored usage of the at least one compression garment 120 in a first state of operation of the compression system 100 when the compression garment 120 is delivering compression to a patient, and a second display indicator 117b, 117d associated with the time of the monitored usage of the garment in a second state of operation of the compression system 100 when the compression garment is not delivering compression to a patient. The indicator unit 117 is configured to automatically swap between the first and second display indicators associated with the states of operation of the compression system 100.

In one aspect, the compression system 100 comprises an identification component 390 associated with a type of the compression garment 120 and mounted on a connector 330 of the compression garment 120. The air pump 110 is configured for regulating air pressure to the compression garment 120 based on the type of the compression garment. The system comprises at least one pressure sensor for measuring the pressure present in at least one of the inflatable chambers of the connected compression garment 120 during a compression therapy and generating signals corresponding to the pressure in the inflatable chambers. The pressure sensor provides signals during the application of compression comprising equivalent characteristics of the presence of a limb or part thereof of a human body located within the at least one compression garment 120. The pressure sensor denotes a change in the compression level of the compression garment 120 as a result of the limb or part thereof of a human body being removed from the compression garment (120) compared to the characteristic measured during the previous inflation. A totalized monitored time when the limb or part thereof of a human body is present in the connected compression garment 120 compared to the monitored time when the limb or anatomical structure is currently detected as not being present in the connected compression garment 120 is monitored and provided by the controller 221 and visually presented to the user on the indicator unit 117.

In one aspect, the identification component 390 is being made of ferrite, steel or brass material.

In one aspect, the first display indicator 117a, 117c includes the totalized duration of time associated with delivery of compression in minutes or in hours.

In one aspect, the second display indicator 117b, 117d includes the duration of time associated with the current lack of delivery of compression in minutes or in hours.

In one aspect, a visible alert is provided when the second display indicator 117b, 117d is active and the lack of delivered compression exceeds a threshold. In aspect, the visible alert comprises the activation of a LED or LCD unit.

In one aspect, the monitored time of usage is configured to be reset by the user.

In one aspect, the first state of operation is associated with correct operation of the compression system 100 and the second state of operation is associated with impaired operation of the compression system 100.

In one aspect, a fluidic output is maintained in both first and second states of operation.

In one aspect, the compression garment 120 is configured for fitment to a limb or part thereof of a human body, where the duration of non-connected time is monitored by the compression system 100 and the time is displayed as the second state of operation and shown as current duration on the indicator unit 117.

In one aspect, the compression garment 120 is configured for fitment to a limb or part thereof of a human body, where the duration of removal from the limb is monitored by the control unit and displayed on the indicator unit 117.

In one aspect, the indicator unit 117 is a LCD display comprising a graphic icon of a limb or part thereof, the icon being flashed or otherwise alternated in contrast when the compression system 100 is in the second state of operation.

In one aspect, the indicator unit 117 is a LCD display providing an indication of product non-usage over time and displayed initially in minutes.

In one aspect, the indicator unit 117 is configured to provide an indication of product non-usage over time and to provide a progressive response as the monitored non-usage over time increases.

In one aspect, a compression garment 120 is provided for fitment to a limb or part thereof of a human body, where the duration of connected time is monitored by a system 100 for monitoring and controlling fluid pressure during compression therapy such that the time in a first state of operation is totalized and displayed on a user interface 117 of an air pump 110 of the compression system 100.

In one aspect, a compression system 100 is provided for monitoring and controlling fluid pressure during compression therapy. The system comprises at least one compression garment 120, an air source 110 for regulating air pressure to at least one compression garment 120 when connected to the air source, a controller operatively connected to the air source 110 and configured for monitoring the usage of the at least one compression garment 120, a limb detection unit. The at least one compression garment 120 comprises a connection unit 330 with an identification component 390, wherein the identification component 390 being made of ferrite, steel or brass. The limb detection unit is configured by the compression system 100 based on the nature of the identification component 390 of the compression garment.

In one aspect, the system comprises a limb detection algorithm, the algorithm including at least one parameter, wherein the parameter being set by the identification component 390 of the at least one compression garment 120 when connected to the compression system 100.

In one aspect, the system comprises two compression garment connection ports 310, wherein the limb detection algorithm is applied to each compression garment connection port separately and independently based on a value of the identification component 390 when attached to each compression connection garment port 310.

In one aspect, a method is provided for monitoring and controlling fluid pressure during compression therapy in a compression system 100. The system 100 comprises a pump 110, at least one inflatable or deflatable compression garment 120, an indicator unit 117, a control unit for monitoring the usage of the at least one compression garment 120. The indicator unit 117 comprises a first display indicator 117a, 117c associated with the time of the monitored usage of the at least one compression garment 120 in a first state of operation of the compression system 100 when the compression garment 120 is delivering compression to a patient, and a second display indicator 117b, 117d associated with the timed of the monitored usage of the garment in a second state of operation of the compression system 100) when the compression garment is not delivering compression to a patient. The method comprises starting 1 the compression system, identifying 2 connected compression garments, starting 4 prophylaxis operation, detecting and displaying 5 the usage of the at least one compression garment 120 of a compression system 100 and automatically swapping 5 between the first and second display indicators 117a, 117b, 117c, 117d associated with the states of operation of the compression system 100.

In one aspect, the method further comprises sensing the presence of a limb or part thereof of a human body within the at least one compression garment 120, and the step of detecting and displaying 5 comprises comparing a first set of sensed data with subsequent sets of sensed data, wherein the differences between the first and the subsequent sets of sensed data being compared with a threshold set by an identification component 390 of the at least one compression garment 120.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms used herein should be interpreted as having a meaning that is consistent with their meaning in the context of this specification and the relevant art and will not be interpreted in an idealized or overly formal sense unless expressly so defined herein.

The foregoing has described the principles, preferred embodiments and modes of operation of the present invention. However, the invention should be regarded as illustrative rather than restrictive, and not as being limited to the particular embodiments discussed above. The different features of the various embodiments of the invention can be combined in other combinations than those explicitly described. It should therefore be appreciated that variations may be made in those embodiments by those skilled in the art without departing from the scope of the present invention as defined by the following claims.

1. A compression system for monitoring and controlling fluid pressure during compression therapy comprising:
at least one inflatable or deflatable compression garment;
an air pump for regulating air pressure to the compression garment when connected to the air pump;
an indicator unit; and
a controller operatively connected to the air pump for monitoring the usage of the at least one compression garment;
wherein the indicator unit comprises:
a first display indicator associated with the time of the monitored usage of the at least one compression garment in a first state of operation of the compression system when the compression garment is delivering compression to a patient, and
a second display indicator associated with the time of the monitored usage of the garment in a second state of operation of the compression system when the compression garment is not delivering compression to a patient, and
wherein the indicator unit is configured to automatically swap between the first and second display indicators associated with the states of operation of the compression system.

2. The system according to claim 1, comprising:
an identification component associated with a type of the compression garment and mounted on a connector of the compression garment, wherein the air pump is configured for regulating air pressure to the compression garment based on the type of the compression garment, and
at least one pressure sensor for measuring the pressure present in at least one of the inflatable chambers of the connected compression garment during a compression therapy and generating signals corresponding to the pressure in the inflatable chambers,
wherein the pressure sensor providing signals during the application of compression comprising equivalent characteristics of the presence of a limb or part thereof of a human body located within the at least one compression garment,
wherein the pressure sensor denotes a change in the compression level of the compression garment as a result of the limb or part thereof of a human body being removed from the compression garment compared to the characteristic measured during the previous inflation, and
wherein a totalized monitored time when the limb or part thereof of a human body is present in the connected compression garment compared to the monitored time when the limb or anatomical structure is currently detected as not being present in the connected compression garment is monitored and provided by the controller and visually presented to the user on the indicator unit.

3. The system according to claim 2, wherein the identification component is being made of ferrite, steel or brass material.

4. The system according to claim 1, wherein the first display indicator includes the totalized duration of time associated with delivery of compression in minutes.

5. The system according to claim 1, wherein the first display indicator includes the totalized duration of time associated with delivery of compression in hours.

6. The system according to claim 1, wherein the second display indicator includes the duration of time associated with the current lack of delivery of compression in minutes.

7. The system according to claim 1, wherein the second display indicator includes the duration of time associated with the lack of delivery of compression in hours.

8. The system according to claim 1, wherein a visible alert is provided when the second display indicator is active and the lack of delivered compression exceeds a threshold.

9. The system according to claim 8, wherein the visible alert comprises the activation of a LED or LCD unit.

10. The system according to claim 1, wherein the monitored time of usage is configured to be reset by the user.

11. The system according to claim 1, wherein the first state of operation is associated with correct operation of the compression system and the second state of operation is associated with impaired operation of the compression system.

12. The system according to claim 1, where a fluidic output is maintained in both first and second states of operation.

13. The system according to claim 1, wherein the compression garment is configured for fitment to a limb or part thereof of a human body, where the duration of non-connected time is monitored by the compression system and the time is displayed as the second state of operation and shown as current duration on the indicator unit.

14. The system according to claim 1, wherein the compression garment is configured for fitment to a limb or part thereof of a human body, where the duration of removal from the limb is monitored by the control unit and displayed on the indicator unit.

15. The system according to claim 1, wherein the indicator unit is a LCD display comprising a graphic icon of a limb or part thereof, the icon being flashed or otherwise alternated in contrast when the compression system is in the second state of operation.

16. The system according to claim 1, wherein the indicator unit is a LCD display providing an indication of product non-usage over time and displayed initially in minutes.

17. The system according to claim 1, wherein the indicator unit is configured to provide an indication of product non-usage over time and to provide a progressive response as the monitored non-usage over time increases.

18. A compression garment for fitment to a limb or part thereof of a human body, where the duration of connected time is monitored by a system for monitoring and controlling fluid pressure during compression therapy such that the time in a first state of operation is totalized and displayed on a user interface of an air pump of the compression system.

19. A compression system for monitoring and controlling fluid pressure during compression therapy, comprising:
at least one compression garment;
an air source for regulating air pressure to at least one compression garment when connected to the air source;
a controller operatively connected to the air source and configured for monitoring the usage of the at least one compression garment; and
a limb detection unit,
wherein the at least one compression garment comprising a connection unit with an identification component,
wherein the identification component being made of ferrite, steel or brass, and
wherein the limb detection unit is configured by the compression system based on the nature of the identification component of the compression garment.

20. The system according to claim 19, comprising a limb detection algorithm, the algorithm including at least one parameter, wherein the parameter being set by the identification component of the at least one compression garment when connected to the compression system.

21. The system according to claim 20, comprising two compression garment connection ports, wherein the limb detection algorithm is applied to each compression garment connection port separately and independently based on a value of the identification component when attached to each compression connection garment port.

22. A method for monitoring and controlling fluid pressure during compression therapy in a compression system, wherein the system comprises a pump, at least one inflatable or deflatable compression garment, an indicator unit, a control unit for monitoring the usage of the at least one compression garment, wherein the indicator unit comprises:
a first display indicator associated with the time of the monitored usage of the at least one compression garment in a first state of operation of the compression system when the compression garment is delivering compression to a patient, and
a second display indicator associated with the timed of the monitored usage of the garment in a second state of operation of the compression system when the compression garment is not delivering compression to a patient, wherein the method comprises:
starting the compression system,
identifying connected compression garments,
starting prophylaxis operation,
detecting and displaying the usage of the at least one compression garment of a compression system, and
automatically swapping between the first and second display indicators associated with the states of operation of the compression system.

23. The method according to claim 22, wherein the method further comprises sensing the presence of a limb or part thereof of a human body within the at least one compression garment, and
wherein the step of detecting and displaying comprises:
comparing a first set of sensed data with subsequent sets of sensed data, wherein the differences between the first and the subsequent sets of sensed data being compared with a threshold set by an identification component of the at least one compression garment.
